(19) United States
(12) Patent Application Publication
(10) Pub. No.: US 2022/0192942 A1
(43) Pub. Date: Jun. 23, 2022

(54) PORTION UNIT WITH COSMETIC AGENT FOR LIGHTENING KERATINOUS FIBERS, ESPECIALLY HUMAN HAIR

(71) Applicant: Henkel AG & Co. KGaA, Duesseldorf (DE)

(72) Inventors: Udo ERKENS, Willich (DE); Torsten LECHNER, Langenfeld (DE)

(73) Assignee: Henkel AG & Co. KGaA, Duesseldorf (DE)

(21) Appl. No.: 17/553,172

(22) Filed: Dec. 16, 2021

(30) Foreign Application Priority Data
Dec. 18, 2020 (DE) 102020134098.2

(51) Int. Cl. A61K 8/23; A61K 8/02; A61K 8/22; A61K 8/81; A61Q 5/08

(52) U.S. Cl. CPC A61K 8/23; A61K 8/0208; A61Q 5/08; A61K 8/8129; A61K 8/22

(57) ABSTRACT

The present disclosure relates to a portion unit having at least one receiving chamber filled with a cosmetic agent for lightening keratinous fibers, in particular human hair, which receiving chamber is formed by a water-soluble envelope made of a water-soluble nonwoven fabric.

### CROSS-REFERENCE TO RELATED APPLICATION

This application claims priority to German Patent Application No. 102020134098.2, filed Dec. 18, 2020, which is incorporated herein by reference in its entirety.

### TECHNICAL FIELD

The present disclosure relates to a portion unit having at least one receiving chamber filled with a cosmetic agent for lightening keratinous fibers, in particular human hair, which receiving chamber is formed by a water-soluble envelope made of a water-soluble nonwoven fabric.

### BACKGROUND

A wide variety of products are available to the consumer for agents to lighten keratin fibers, especially human hair. Conventional agents, so-called bleaching powders, are portioned by employing a spoon before application. This causes dust for the consumer, which should be avoided. In addition, the portion size is variable, as the spoon may be filled differently, giving different results with the same product.

There is therefore a need for easy handling of bleaching powders, i.e., agents for lightening keratin fibers, especially human hair.

### BRIEF SUMMARY

This disclosure provides a portion unit comprising at least one receiving chamber formed by a water-soluble envelope, wherein the water-soluble envelope comprises a water-soluble nonwoven fabric and wherein the receiving chamber comprises a cosmetic agent for lightening keratinous fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it was found that this task is solved by providing an agent for lightening keratinous fibers, in particular human hair, in a portion unit formed from a water-soluble nonwoven fabric.

Therefore, a first object of the present disclosure is a portion unit comprising at least one receiving chamber formed by a water-soluble envelope, wherein the water-soluble envelope comprises or consists of a water-soluble nonwoven fabric and wherein the receiving chamber comprises a cosmetic agent for lightening keratinous fibers, in particular human hair.

Prefabrication can avoid the formation of dust when using the bleaching powder, i.e., the agent for lightening keratin fibers, especially human hair. In addition, the sachet provides a defined application amount, minimizing misapplication due to incorrect mixing ratios. The sachet as contemplated herein is only mixed with a developer containing hydrogen peroxide before application and can be applied after reaching a sufficient application viscosity. In storage tests with hygroscopic blonding powder, the nonwoven proved to be more storage stable than commercially available water-soluble films. In the case of water-soluble PVA films, storage tests led to embrittlement of the film, which was reflected in a reduced solubility behavior of the film. In addition, it also leads to increased fragility of the sachet. These disadvantages are also solved by the portioning unit as contemplated herein.

By providing the cosmetic agent in a water-soluble wrapper as contemplated herein, simple and uncomplicated handling of the agent is made possible while avoiding additional packaging waste. This allows the agent to be easily converted to a ready-to-use state without the need for additional portioning. In addition, by using its nonwoven material as the wrapping/portioning unit, the end customer has a tactile soft feeling when using it.

### Water Soluble Coating

The portion unit as contemplated herein comprises or consists of at least one receiving chamber formed by a water-soluble envelope comprising or consisting of a water-soluble nonwoven fabric.

The term nonwoven is used to describe structures made of fibers that are joined together to form a fiber layer. The nonwovens are preferably flexible and pliable. Films or papers are not counted as nonwovens.

In a preferred embodiment of the portion units, the water-soluble nonwovens are needled nonwovens. In corresponding nonwovens, entanglement and thus compaction and consolidation of the fibers is achieved by needling, for example by employing suitable needle boards or needle bars.

Preferred nonwovens, preferably needle-punched nonwovens, are those from the group of random nonwovens. In these nonwovens, the fibers have any direction and are evenly distributed in all directions of the nonwoven. Corresponding nonwovens are not only exemplified by a special look and feel, but also improve the mechanical stability of the cosmetic product encompassed by the portion unit during transport and storage due to their compressibility.

Regarding the optical, haptic and mechanical properties of the portion unit, it has proved advantageous if the water-soluble nonwoven, in particular the random nonwoven, has a basis weight of from about 20 to about 200 g/m², preferably from about 30 to about 100 g/m² and from about 35 to about 70 g/m². A higher basis weight results in a higher stability of the coating but reduces the solubility in water or prolongs the duration of the dissolution of the coating.

Due to their properties, water-soluble nonwovens from the group of cross-layer nonwovens are particularly preferred in addition to the random-layer nonwovens. These nonwovens are produced, for example, by laying down the fibers on a laying-down belt with a longitudinal orientation of the fibers to the overall nonwoven, the fibers preferably being oriented in two directions.

Particularly preferably, the water-soluble nonwoven fabric is a non-woven, cross-laid and spot-sealed nonwoven fabric, which is preferably single-ply. When using such cross laid nonwovens, it has proved advantageous if the water-soluble nonwoven, the cross laid nonwoven, has a basis weight of about 20 to about 80 g/m², preferably about 35 to about 70 g/m², particularly preferably about 38 to about 60 g/m².

In a further preferred embodiment, the water-soluble nonwoven fabric is a woven nonwoven fabric. When using a woven water-soluble nonwoven, very satisfactory results were obtained, particularly regarding the mechanical strength and tightness of the portion unit. In embodiments in which a nonwoven fabric is used, the nonwoven fabric preferably has a basis weight of about 35 to about 160 g/m², preferably about 80 to about 120 g/m² or about 35 to about 80 g/m² or about 120 to about 160 g/m².

In another preferred embodiment of the portion unit, the water-soluble nonwovens are cloth-knit nonwovens. In these nonwovens, the bonding of the fibers is achieved by fabric closure using additives. These binders enable the fibers to be adhesively bonded to each other. Preferred nonwovens are based on water-soluble polymer fibers, in particular water-soluble fibers based on polyvinyl alcohol and polyvinyl alcohol copolymers. In a preferred embodiment, the water-soluble nonwoven comprises at least about 50 wt. %, preferably at least about 70 wt. % and in particular at least about 98 wt. % of polyvinyl alcohol or polyvinyl alcohol copolymers, in particular polyvinyl alcohol, the weight percentages in each case being based on the total weight of the water-soluble coating. In addition to good water solubility, fibers based on polyvinyl alcohols and polyvinyl alcohol copolymers have the advantage that they are biodegradable and can thus be returned to the value-adding cycle. The degree of saponification of the polyvinyl alcohol is preferably about 80 to about 100%, preferably about 84 to about 99%, more preferably about 84 to about 98%, more preferably about 90 to about 97%, more preferably about 91 to about 96%, more preferably about 92 to about 95%, more preferably about 93 to about 94%.

The fiber length of the water-soluble polymer fibers is preferably in the range from about 1 to about 40 mm, particularly preferably from about 2 to about 20 mm. The thickness of the fibers is preferably about 0.1 to about 1000 μm, preferably about 0.5 to about 400 μm. The density of the nonwoven is preferably above about 0.15 g/cm³, preferably above about 0.2 g/cm³ and in the range of about 0.2 to about 0.8 g/cm³.

The nonwovens can be produced, for example, by employing mechanical, aerodynamic or hydrodynamic processes of the prior art. The fibers of the nonwoven can be bonded mechanically, for example by frictional or positive locking, chemically, for example using binders or solvents, or thermally, using thermoplastic additives.

To improve the stability and tightness of the portion unit, the water-soluble wrapping of the portion unit as contemplated herein preferably further comprises a water-soluble polymer film, the film preferably being a film of polyvinyl alcohol. In a particularly preferred embodiment, the water-soluble nonwoven fabric is coated with the water-soluble film, in particular the side of the nonwoven fabric that meets the cosmetic agent during intended use. In this way, the tightness of the portion unit as contemplated herein can be further improved so that the portion unit can also be used and stored without problems in connection with liquid or gel cosmetic products.

There are no specific requirements for the shape and size of the portion unit. Rather, the shape and size can be adjusted as needed. In a preferred embodiment, the portion unit is formed entirely by the receiving chamber. In an alternative preferred embodiment, the portion unit has several, preferably separate, receiving chambers.

The surface of the portion unit can be used to make the portion unit visually appealing. Thus, in a preferred embodiment, the portion unit has a printed surface.

### Cosmetic Agent for Lightening Keratinous Fibers

The cosmetic agent included in the portion unit as contemplated herein is one for lightening keratin fibers, in particular human hair. The agent is preferably in solid form, especially in powder form.

As contemplated herein, the term “powder” or “powdery” means a solid, free-flowing dosage form comprising individual particles at about 20° C. and about 1013 mbar, in which the individual particles have particle sizes in the range from about 0.1 μm to a maximum of about 1.6 mm. The determination of particle sizes can preferably be done by laser diffraction measurement according to ISO 13320-1 (2009).

The cosmetic agent for lightening keratin fibers, in particular human hair, included in the portion unit as contemplated herein is anhydrous, i.e., it has a water content of 0 to 8 wt. %, preferably about 0.1 to about 5 wt. %, particularly preferably about 0.5 to about 2.5 wt. %, based on its weight, as contemplated herein. With the above-mentioned water contents in the range from about 0 to about 8 wt. %, the cosmetic product portioned as contemplated herein is anhydrous within the meaning of the present application. These data refer to the free water content. The content of molecularly bound water or water of crystallization, which individual powder components may have, is not considered. The water content can be measured with a moisture analyzer or moisture meter, for example a moisture meter from Mettler, model Mettler HS 153, where the loss on drying is determined at about 105° C., shut-off criterion about 50 seconds with a product weight of about 1 to about 1.5 grams.

The cosmetic composition included in the portion unit as contemplated herein comprises at least one hair-lightening oxidizing agent selected from peroxodisulfates, percarbonates, perborates and percarbamide, as well as mixtures of these substances.

To use the cosmetic composition as contemplated herein, it must be brought into contact—depending on the oxidizing agent used—with an aqueous hydrogen peroxide preparation or only with water in the absence of hydrogen peroxide, so that the effect, namely the lightening of keratinous fibers, especially human hair, occurs.

When peroxodisulfate(s) or peroxodisulfate-containing mixtures are used, the ready-to-use hair lightening agent is prepared by mixing the peroxodisulfate-containing portion unit of the present disclosure with an aqueous hydrogen peroxide preparation. When percarbonates, perborates or percarbamide are used, the hair lightening composition ready for use is prepared by mixing the portion unit comprising percarbonate or perborate or percarbamide as contemplated herein with water, for example tap water, or an aqueous preparation free from hydrogen peroxide.

In a first embodiment of the present disclosure, the cosmetic composition included in the portion unit as contemplated herein comprises, based on its total weight, about 10 to about 90 wt. % of peroxodisulfate(s) as an essential component. Preferred is the use of about 20 to about 80 wt. % and about 30 to about 60 wt. % peroxodisulfate(s).

Preferred peroxodisulfates to be used are the alkali and ammonium peroxodisulfates, sodium peroxodisulfate, potassium peroxodisulfate, ammonium peroxodisulfate and mixtures thereof. The weight ratios of the different peroxodisulfates are freely selectable but can also be set within specified limits. For example, the amount of potassium peroxodisulfate can always be kept larger than the amount of sodium and ammonium peroxodisulfate that may be used. By way of example, this is illustrated by an embodiment of cosmetic compositions as contemplated herein which comprise, based on their total weight, from about 5 to about 50 wt. %, preferably from about 5 to about 40 wt. %, more preferably from about 10 to about 30 wt. %, of potassium peroxodisulfate, and the weight ratio of potassium peroxodisulfate included in the composition to the total amount of peroxodisulfates included in the composition is at least about 0.5, preferably at least about 0.7 and more preferably at least about 0.9.

In a preferred embodiment, the cosmetic composition comprises potassium peroxodisulfate, ammonium peroxodisulfate and sodium peroxodisulfate. Particularly preferably, it comprises potassium peroxodisulfate in a proportion of, based on the total weight of the agent in the coating, about 5 to about 50 wt. %, preferably about 5 to about 40 wt. %, more preferably about 10 to about 30 wt. % and/or ammonium peroxodisulfate in a proportion of, based on the total weight of the agent in the coating, about 5 to about 30 wt.-%, preferably about 5 to about 20 wt. %, more preferably about 5 to about 15 wt. % and/or sodium peroxodisulfate in a proportion of, based on the total weight of the agent in the coating, about 2 to about 30 wt. %, preferably about 5 to about 20 wt. %, more preferably about 6 to about 8 wt. %.

In another embodiment of the present disclosure, wherein the portioned hair lightening agent can be processed into a ready-to-use hair lightening agent by mixing with water without hydrogen peroxide, the portioned agent comprises, based on its total weight, at least one member selected from the group of percarbamide, percarbonates and perborates in a total amount of about 0.5 to about 95 wt. %. The solid oxidant preparation thus comprises as oxidant percarbamide (addition compound of hydrogen peroxide and urea), at least one percarbonate, sodium percarbonate (2Na₂CO₃.3H₂O₂) and/or at least one perborate, in particular sodium perborate, in a total amount of about 0.5 to about 95 wt. %, preferably about 30 to about 60 wt. %, more preferably about 35 to about 55 wt. %, even more preferably about 40 to about 50wt. %, based on its total weight.

A portioned hair lightening composition preferred as contemplated herein comprises, based on its weight, about 0.5 to about 95 wt. %, preferably about 30 to about 60 wt. %, more preferably about 35 to about 55 wt. %, still more preferably about 40 to about 50 wt. %, of sodium percarbonate.

A further portioned hair lightening agent preferred as contemplated herein comprises, based on its weight, from about 0.5 to about 95 wt. %, preferably from about 30 to about 60 wt. %, more preferably from about 35 to about 55 wt. %, still more preferably from about 40 to about 50 wt. %, of sodium perborate. A further portioned hair lightening agent preferred as contemplated herein comprises, based on its weight, from about 0.5 to about 95wt. %, preferably from about 30 to about 60 wt. %, more preferably from about 35 to about 55 wt. %, still more preferably from about 40 to about 50 wt. %, of percarbamide.

Where quantity ranges are indicated in the present application, the end ranges mentioned are included in each case. Intermediate values are also revealed.

The portioned cosmetic composition for lightening keratinous fibers as contemplated herein comprises, in addition to the at least one oxidizing agent selected from peroxodisulfates, percarbonates, perborates and percarbamide, preferably further at least one alkalizing agent selected from sodium silicate, sodium metasilicate, sodium orthosilicate, sodium disilicate and sodium trisilicate and mixtures thereof. This alkalizing agent enhances the lightening effect of the oxidizing agent in the ready-to-use hair lightener. Surprisingly, it has been found to be beneficial for the storage stability and dissolution properties of the cosmetic compositions to use at least one alkalizing agent selected from sodium silicates and sodium metasilicates, each having a molar SiO₂/Na₂O ratio of from about 0.8 to about 3.7, preferably from about 1.0 to about 3.6, more preferably from about 2.5 to about 3.5.

The total amount of the at least one alkalizing agent selected from sodium silicate, sodium metasilicate, sodium orthosilicate, sodium disilicate and sodium trisilicate and mixtures thereof in the cosmetic composition is preferably in the range from about 0% to about 40 wt. %, from about 2% to about 30 wt. %, preferably from about 5% to about 25 wt. %, extremely preferably from about 10% to about 20 wt. %, in each case based on the weight of the cosmetic composition.

In addition to the oxidizing agents and sodium silicate-based alkalizing agents described above, preferred cosmetic agents for lightening keratin fibers comprise basic magnesium carbonate (magnesium carbonate hydroxide) as a secondary alkalizing agent. Basic magnesium carbonate (magnesium carbonate hydroxide) has several advantages in the context of the present disclosure: it has a weaker alkaline effect than the sodium silicate-based alkalizing agents described above, in particular the sodium silicates and sodium metasilicates, and thus buffers the alkalinity of the hair lightening agent ready for use. Adjusting the preferred pH value of the ready-to-use hair lightening agent as contemplated herein with one of the sodium (meta)silicates alone can lead to undesirable foaming when preparing the application mixture. This is slowed down by the addition of basic magnesium carbonate (magnesium carbonate hydroxide). In addition, the ready-to-use hair lightening agent forms a fine creamy mass in the presence of basic magnesium carbonate (magnesium carbonate hydroxide). Preferred cosmetic hair lightening agents comprise, in each case based on their weight, about 0.5- about 40 wt. %, preferably 1-30 wt. %, particularly preferably about 5- about 20 wt. %, exceptionally preferably about 10- about 15 wt. % basic magnesium carbonate (magnesium carbonate hydroxide).

In a further preferred embodiment, the hair lightening composition portioned as contemplated herein comprises at least one excipient selected from fillers, anti-caking agents and drying agents and mixtures of these excipients. These excipients are intended to prevent the powder components of the cosmetic product from clumping or caking.

Particularly preferred fillers are selected from sodium sulphate and sodium chloride and mixtures thereof. Sodium chloride is particularly preferred because it can enhance the oxidizing effect of the oxidizing agent, especially sodium percarbonate. This is particularly desirable for achieving light color tones. The content of sodium chloride in the cosmetic composition is preferably about 0.1- about 5 wt. %, preferably about 0.2- about 4 wt. %, more preferably about 0.3- about 3 wt. %, still more preferably about 0.4- about 1.5 wt. %-%, most preferably more than about 0.5 to less than about 0.7 wt. %, in each case based on the weight of the cosmetic composition portioned as contemplated herein.

Particularly preferred anti-caking agents are selected from pyrogenic silicas, diatomaceous earth, calcium phosphate, calcium silicates, aluminum oxide, magnesium oxide, magnesium carbonate, zinc oxide, stearates and mixtures thereof.

Particularly preferred desiccants are selected from sodium sulfate, sodium carbonate, magnesium sulfate, calcium chloride and fumed silica, and mixtures thereof. Cosmetic compositions preferred as contemplated herein comprise at least one auxiliary selected from fillers, anti-caking agents and drying agents and mixtures of these auxiliaries in a total amount of about 0.5- about 80 wt. %, preferably about 5- about 65 wt. %, preferably from about 10 to about 55 wt. %, from about 20 to about 35 wt. %, based on the weight of the cosmetic composition.

### Multi-Chamber Portioning Unit

In a further preferred embodiment, the wrapper as contemplated herein may be in the form of a multi-chamber portion unit which comprises the cosmetic composition per se in one chamber of the nonwoven wrapper and further ingredients in a second and optionally third chamber. This wrapping variant is suitable for ingredients that cannot be stably stored in direct contact with, for example, the solid oxidizing agent or the alkalizing agent.

For application, depending on the oxidizing agent used, the cosmetic product is brought into contact with an aqueous hydrogen peroxide preparation or only with water or an aqueous preparation in the absence of hydrogen peroxide, resulting in a ready-to-use mixture. For this purpose, the envelope comprising the cosmetic agent may be placed in a bowl and water or the aqueous hydrogen peroxide preparation added as explained above. Stirring produces an application mixture, which can be applied with a brush (brush application). The envelope(s) can also be placed in a bottle and water, or the aqueous hydrogen peroxide preparation is poured into it. Shaking then creates the application mixture, which can be applied to the keratinous fibers.

To prevent the ready-to-use hair lightening agent from running or flowing away from the active site during the application period, the application mixtures have a viscosity of from about 5 to about 100 Pa·s, preferably from about 10 to about 50 Pa·s, particularly from about 10 to about 20 Pa·s, and more preferably from about 10 to about 16 Pa·s (Brookfield, 22° C., spindle #5, 4 rpm). For this purpose, preferred cosmetic compositions comprise at least one thickening agent and/or at least one gelling agent. Both inorganic and organic substances are suitable as thickeners or gelling agents.

The thickening agent can be selected, for example, from the polymeric thickening agents known under the following INCI names: Acrylamides Copolymer, Acrylamide/Sodium Acrylate Copolymer, Acrylamide/Sodium Acryloyldimethyltaurate Copolymer, Acrylates/Acetoacetoxyethyl Methacrylate Copolymer, Acrylates/Beheneth-25 Methacrylate Copolymer, Acrylates/C 10-30 Alkyl Acrylate Crosspolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-25 Acrylate Copolymer, Acrylates/Palmeth-25 Itaconate Copolymer, Acrylates/Steareth-50 Acrylate Copolymer, Acrylates/Steareth-20 Itaconate Copolymer, Acrylates/Steareth-20 Methacrylate Copolymer, Acrylates/Stearyl Methacrylate Copolymer, Acrylates/Vinyl Isodecanoate Crosspolymer, Acrylic Acid/Acrylonitrogens Copolymer, Agar, Agarose, Alcaligenes Polysaccharides, Algin, Alginic Acid, Ammonium Acrylates/Acrylonitrogens Copolymer, Ammonium Acrylates Copolymer, Ammonium Acryloyldimethyltaurate/Vinyl Formamide Copolymer, Ammonium Acryloyldimethyltaurate/VP Copolymer, Ammonium Alginate, Ammonium Polyacryloyldimethyl Taurate, Amylopectin, Ascorbyl Methylsilanol Pectinate, Astragalus Gummifer Gum, Attapulgite, Avena Sativa (Oat) Kernel Flour, Bentonite, Butoxy Chitosan, Caesalpinia Spinosa Gum, Calcium Alginate, Calcium Carboxymethyl Cellulose, Calcium Carrageenan, Calcium Potassium Carbomer, Calcium Starch Octenylsuccinate, C20-40 Alkyl Stearate, Carbomer, Carboxybutyl Chitosan, Carboxymethyl Chitin, Carboxymethyl Chitosan, Carboxymethyl Dextran, Carboxymethyl Hydroxyethylcellulose, Carboxymethyl Hydroxypropyl Guar, Cellulose Acetate Propionate Carboxylate, Cellulose Gum (Sodium Carboxymethylcellulose), Cetyl Hydroxyethylcellulose, Cholesterol/HDI/Pullulan Copolymer, Cholesteryl Hexyl Dicarbamate Pullulan, Cyamopsis Tetragonoloba (Guar) Gum, Diglycol/CHDM/lsophthalates/SIP Copolymer, Dihydrogenated Tallow Benzylmonium Hectorite, Dimethicone Crosspolymer-2, Dimethicone Propyl PG-Betaine, DMAPA Acrylates/Acrylic Acid/Acrylonitrogens Copolymer, Ethylene/Sodium Acrylate Copolymer, Gelatin, Gellan Gum, Glyceryl Alginate, Glycine Soja (Soybean) Flour, Guar Hydroxypropyltrimonium Chloride, Hectorite, Hydrated Silica, Hydrogenated Potato Starch, Hydroxybutyl Methylcellulose, Hydroxyethyl Acrylate/Sodium Acryloyldimethyl Taurate Copolymer, Hydroxyethylcellulose, Hydroxyethyl Chitosan, Hydroxyethyl Ethylcellulose, Hydroxypropylcellulose, Hydroxypropyl Chitosan, Hydroxypropyl Ethylenediamine Carbomer, Hydroxypropyl Guar, Hydroxypropyl Methylcellulose, Hydroxypropyl Methylcellulose Stearoxy Ether, Hydroxystearamide MEA, Isobutylene/Sodium Maleate Copolymer, Lithium Magnesium Silicate, Lithium Magnesium Sodium Silicate, Macrocystis Pyrifera (Kelp), Magnesium Alginate, Magnesium Aluminum Silicate, Magnesium Silicate, Magnesium Trisilicate, Methoxy PEG-22/Dodecyl Glycol Copolymer, Methylcellulose, Methyl Ethylcellulose, Methyl Hydroxyethylcellulose, Microcrystalline Cellulose, Montmorillonite, Moroccan Lava Clay, Natto Gum, Nonoxynyl Hydroxyethylcellulose, Octadecene/MA Copolymer, Pectin, PEG-800, PEG Crosspolymer, PEG-150/Decyl Alcohol/SMDI Copolymer, PEG-175 Diisostearate, PEG-190 Distearate, PEG-15 Glyceryl Tristearate, PEG-140 Glyceryl Tristearate, PEG-240/HDI Copolymer Bis-Decyltetradeceth-20 Ether, PEG-100/IPDI Copolymer, PEG-180/Laureth-50/TMMG Copolymer, PEG-10/Lauryl Dimethicone Crosspolymer, PEG-15/Lauryl Dimethicone Crosspolymer, PEG-2M, PEG-5M, PEG-7M, PEG-9M, PEG-14M, PEG-20M, PEG-23M, PEG-25M, PEG-45M, PEG-65M, PEG-90M, PEG-1 15M, PEG-160M, PEG-120 Methyl Glucose Trioleate, PEG-180/Octoxynol-40/TMMG Copolymer, PEG-150 Pentaerythrityl Tetrastearate, PEG-4 Rapeseedamide, PEG-150/Stearyl Alcohol/SMDI Copolymer, Polyacrylate-3, Polyacrylic Acid, Polycyclopentadiene, Polyether-1, Polyethylene/Isopropyl Maleate/MA Copolyol, Polymethacrylic Acid, Polyquaternium-52, Polyvinyl Alcohol, Potassium Alginate, Potassium Aluminum Polyacrylate, Potassium Carbomer, Potassium Carrageenan, Potassium Polyacrylate, Potato Starch Modified, PPG-14 Laureth-60 Hexyl Dicarbamate, PPG-14 Laureth-60 Isophoryl Dicarbamate, PPG-14 Palmeth-60 Hexyl Dicarbamate, Propylene Glycol Alginate, PVP/Decene Copolymer, PVP Montmorillonite, Rhizobian Gum, Ricinoleic Acid/Adipic Acid/AEEA Copolymer, Sclerotium Gum, Sodium Acrylate/Acryloyldimethyl Taurate Copolymer, Sodium Acrylates/Acrolein Copolymer, Sodium Acrylates/Acrylonitrogens Copolymer, Sodium Acrylates Copolymer, Sodium Acrylates/Vinyl Isodecanoate Crosspolymer, Sodium Acrylate/Vinyl Alcohol Copolymer, Sodium Carbomer, Sodium Carboxymethyl Chitin, Sodium Carboxymethyl Dextran, Sodium Carboxymethyl Beta-Glucan, Sodium Carboxymethyl Starch, Sodium Carrageenan, Sodium Cellulose Sulfate, Sodium Cyclodextrin Sulfate, Sodium Hydroxypropyl Starch Phosphate, Sodium Isooctylene/MA Copolymer, Sodium Magnesium Fluorosilicate, Sodium Polyacrylate, Sodium Polyacrylate Starch, Sodium Polyacryloyldimethyl Taurate, Sodium Polymethacrylate, Sodium Polystyrene Sulfonate, Sodium Silicoaluminate, Sodium Starch Octenylsuccinate, Sodium Stearoxy PG-Hydroxyethylcellulose Sulfonate, Sodium Styrene/Acrylates Copolymer, Sodium Tauride Acrylates/Acrylic Acid/Acrylonitrogens Copolymer, Solanum Tuberosum (Potato) Starch, Starch/Acrylates/Acrylamide Copolymer, Starch Hydroxypropyltrimonium Chloride, Steareth-60 Cetyl Ether, Steareth-100/PEG-136/HDI Copolymer, Sterculia Urens Gum, Synthetic Fluorphlogopite, Tamarindus Indica Seed Gum, Tapioca Starch, TEA Alginate, TEA Carbomer, Triticum Vulgare (Wheat) Starch, Tromethamine Acrylates/Acrylonitrogens Copolymer, Tromethamine Magnesium Aluminum Silicate, Welan Gum, Xanthan Gum, Yeast Beta-Glucan, Yeast Polysaccharides, Zea mays (Com) Starch.

Preferably, the thickener is selected from xanthan gum, carboxymethyl cellulose, and mixtures thereof.

The at least one polymeric thickener is preferably present in a total amount of from about 0.1 to about 10 wt. %, from about 0.4 to about 5.0 wt. %, in the cosmetic composition portioned as contemplated herein.

### Liquid Oxidizer Preparation

As contemplated herein, if the coating has an agent comprising peroxodisulfate(s), the coating is preferably contacted with a liquid oxidant preparation (M2) for use. Such an oxidant preparation preferably comprises about 40- about 96 wt. %, preferably about 70- about 93 wt. %, particularly preferably about 80- about 90 wt. %, of water, further hydrogen peroxide in a total amount of about 0.5 to about 23 wt. %, further preferably about 2.5 to about 21 wt. %, particularly preferably about 4 to about 20 wt. %, very preferably about 5 to about 18 wt. % and exceptionally preferably about 6 to about 12 wt. %, and has a pH in the range from about 2.0 to about 6.5, preferably about 2.5- about 5.5, particularly preferably about 2.8 to about 5.0, in each case measured at about 20° C., the percentages by weight in each case being based on the weight of the oxidizer preparation (M2).

Optionally, the oxidant preparation (M2) may further comprise at least one cationic surfactant.

For oxidative hair lightening processes, the agent as contemplated herein, which is in the envelope, is usually mixed with the aqueous oxidant-containing composition (oxidant preparation (M2)) immediately before application to the hair to form the ready-to-use lightening agent and then applied to the hair. In most cases, the agent as contemplated herein and the oxidizing agent preparation (M2) are matched to each other in such a way that, at a mixing ratio of about 1 to 1, based on parts by weight, there is an initial concentration of hydrogen peroxide of about 0.5- about 12 wt. %, preferably about 2- about 10 wt. %, particularly preferably about 3- about 6 wt. % hydrogen peroxide (calculated as 100% H₂O₂), in each case based on the weight of the application mixture, in the finished application mixture. However, it is just as well possible to match the agent as contemplated herein and the oxidant preparation (M2) in such a way that the concentrations required in the ready-to-use oxidation whitening agent (application mixture) are obtained by mixing ratios other than 1:1, for example by a weight-based mixing ratio of about 1:2 or about 1:3 or even about 2:3.

Weight-based mixing ratios preferred as contemplated herein (cosmetic agent):(liquid oxidant preparation (M2)) are in the range of about 1:0.8 to about 1:2.5, particularly preferably in the range of about 1:1 to about 1:2.

By portioning the cosmetic agent for lightening keratin-containing fibers, in particular hair, as contemplated herein, the mixing ratio can be well adjusted and controlled. In a preferred embodiment, the oxidizer preparation (M2) is also provided in portions to ensure consistent whitening.

The oxidant preparation (M2) usually has a low viscosity in the range of about 10- about 6000 mPas, preferably about 200- about 5000 mPas, particularly preferred about 1000- about 4500 mPas, each measured at about 20° C. (Brookfield viscometer, rotation frequency of 4 min-1, spindle No. 5). For application to the hair, however, the application mixture should have a significantly higher viscosity so that it remains on the hair during the entire application time (in the range of about 5- about 60 minutes, preferably about 30- about 45 minutes) and does not drip down. The application mixture of cosmetic agent and oxidant preparation (M2) is usually prepared by mixing the portion unit as contemplated herein with the oxidant preparation in a bowl, from which the application mixture is applied to the hair with a brush immediately after mixing (brush application).

Surprisingly, it was found that an application mixture with a viscosity suitable for brush application is obtained when the agent as contemplated herein is mixed in the portion unit with an oxidizing agent preparation (M2) comprising at least one cationic surfactant. During mixing, the interaction between the at least one crosslinked copolymer of acrylic acid and non-ethoxylated esters of acrylic acid with linear C10-C30 monoalcohols and the at least one cationic surfactant leads to the desired increase in viscosity. The resulting paste-like consistency of the application mixture leads to optimal application properties, especially for brush application. The application mixtures obtained in this way, in particular the mixtures whose mixing ratio by weight (cosmetic agent in the coating):(oxidizing agent preparation (M2)) is in the range from about 1:0.8 to about 1:2.5, particularly preferably in the range from about 1:1 to about 1:2, preferably have a viscosity in the range of about 20000- about 100000 mPas, preferably about 30000- about 80000 mPas, particularly preferably about 45000- about 70000 mPas, in each case measured at 20° C. (Brookfield viscometer, rotation frequency of 4 min-1, spindle no. 5).

In a further preferred embodiment of the present disclosure, the oxidant preparation (M2) used as contemplated herein comprises at least one cationic surfactant, preferably in a total amount of about 0.05- about 3wt. %, particularly preferably of about 0.1- about 1.5wt. %, exceptionally preferably of about 0.3- about 0.9wt. %, in each case based on the weight of the oxidant preparation.

Cationic surfactants or cationic surfactants are surfactants, i.e., surface-active compounds, each with one or more positive charges. Cationic surfactants comprise only positive charges. Usually, these surfactants are composed of a hydrophobic part and a hydrophilic head group, the hydrophobic part usually comprising a hydrocarbon backbone (e.g., comprising one or two linear or branched alkyl chains) and the positive charge(s) being in the hydrophilic head group. Cationic surfactants adsorb at interfaces and aggregate in aqueous solution above the critical micelle formation concentration to form positively charged micelles.

As contemplated herein, cationic surfactants of the type of quaternary ammonium compounds, esterquats and alkylamidoamines are preferred. Preferred quaternary ammonium compounds are ammonium halides, such as alkyltrimethylammonium chlorides, dialkyldimethylammonium chlorides, trialkylmethylammonium chlorides, as well as the imidazolium compounds known under the INCI designations Quaternium-27 and Quaternium-83. Other preferred quaternary ammonium compounds are tetraalkylammonium salts, such as in particular the quaternium-52 known under the INCI designation, a poly(oxy-1.2-ethanediyl), ((octadecylnitrilio)tri-2.1-ethanediyl)tris(hydroxy)phosphate (1:1) salt, which has the general structural formula (III), wherein x+y+z=about 10:

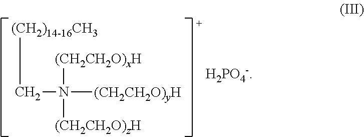

The long alkyl chains of the surfactants mentioned above preferably have about 10 to about 22, particularly preferably about 12 to about 18 carbon atoms. Behenyl trimethylammonium chloride, stearyl trimethylammonium chloride and cetyl trimethylammonium chloride are particularly preferred, with stearyl trimethylammonium chloride being extremely preferred. Further cationic surfactants suitable as contemplated herein are quaternized protein hydrolysates. Alkylamidoamines are usually produced by amidation of natural or synthetic fatty acids and fatty acid cuts with dialkylaminoamines. Tegoamid® S 18 (stearamidopropyldimethylamine) is a suitable compound from this group of substances. Esterquats are substances which comprise both at least one ester function and at least one quaternary ammonium group as a structural element. Preferred esterquats are quaternized ester salts of fatty acids with triethanolamine, quaternized ester salts of fatty acids with diethanolalkylamines and quaternized ester salts of fatty acids with 1.2-dihydroxypropyl dialkylamines. Such products are sold under the Stepantex, Dehyquart and Armocare trademarks.

In terms of optimum application properties and optimum whitening results, C10-C22 alkyl trimethyl ammonium chlorides have proven to be particularly suitable. Particularly preferred oxidizing agent preparations (M2) used as contemplated herein are therefore extremely preferred at least one cationic surfactant in a total amount of about 0.05 to about 3wt. %, particularly preferably about 0.1- about 1.5wt. % from about 0.3 to about 0.9wt. %, based in each case on the weight of the oxidizing agent preparation (M2), preferably at least one surfactant selected from C10-C22-alkyltrimethyl ammonium chlorides, in particular selected from behenyltrimethylammonium chloride, stearyltrimethylammonium chloride and cetyltrimethylammonium chloride, and mixtures of these surfactants. Exceptionally preferred oxidant preparations used as contemplated herein comprise stearyl trimethyl ammonium chloride in a total amount of about 0.05- about 3wt. %, particularly preferred about 0.1- about 1.5wt. %, exceptionally preferred about 0.3- about 0.9wt. %, in each case based on the weight of the oxidant preparation (M2). In a preferred process for oxidative hair lightening as contemplated herein the liquid oxidant preparation (M2) comprises at least one cationic surfactant, preferably in a total amount of about 0.05- about 3 wt. %, particularly preferably of about 0.1- about 1.5 wt. %, extremely preferably of about 0.3- about 0.9 wt. %, in each case based on the weight of the oxidant preparation (M2), but no polymer with a degree of polymerization of at least about 200 and no polymer having a molecular weight of about 10,000 Daltons or higher.

In addition to the so-called brush application, it is also possible to apply the agent in the wrapper as contemplated herein by employing a bottle. For this purpose, the oxidant preparation (M2) is provided in a bottle into which the portion unit is then inserted (so-called bottle application). Thorough shaking dissolves the coating and the cosmetic product meets the oxidant preparation.

Surprisingly, it was found that an application mixture with a viscosity suitable in particular for bottle application is obtained if the oxidant preparation (M2) comprises at least one copolymer selected from crosslinked acrylic acid/acrylic acid C1-C6 alkyl ester copolymers and crosslinked methacrylic acid/acrylic acid C1-C6 alkyl ester copolymers, preferably in a total amount of about 0.1- about 7 wt.-%, particularly preferably about 0.5- about 6 wt. %, exceptionally preferably about 1- about 4.5 wt. %, in each case based on the weight of the oxidizer preparation. Mixing the coating as contemplated herein or the agent included therein with such an oxidant preparation leads to the desired increase in viscosity. The resulting medium viscous consistency of the application mixture leads to optimal application properties, especially for bottle application. The application mixtures obtained in this way, in particular at weight-related mixing ratios (cosmetic agent in the coating):(liquid oxidant preparation (M2)) in the range from about 1:0.8 to about 1:2.5, particularly preferably in the range from about 1:1 to about 1:2, preferably have a viscosity in the range of about 10000- about 50000 mPas, preferably about 15000- about 30000 mPas, particularly preferably about 18000- about 25000 mPas, in each case measured at about 20° C. (Brookfield viscometer, rotation frequency 4 min-1, spindle no. 5).

### Process and Packaging Unit

In a further embodiment, the problem underlying the present disclosure is solved by a method for lightening keratinous fibers, in particular human hair, in which a coating as previously described is dissolved either in water or in a liquid oxidant preparation and the application preparation thus obtained is applied to the keratinous fibers.

Thus, an object of the present disclosure is a cosmetic process for lightening keratinous fibers, in particular human hair, which is exemplified by contacting a portion unit as previously described with a liquid oxidant preparation (M2), wherein the liquid oxidant preparation comprises about 40- about 96 wt. %, preferably about 70- about 93 wt. %, more preferably about 80- about 90 wt. %, of water, further comprises hydrogen peroxide in a total amount of about 0.5 to about 23 wt.-wt. %, further preferably about 2.5 to about 21 wt. %, particularly preferably about 4 to about 20 wt. %, most preferably about 5 to about 18 wt. % and exceptionally preferably about 6 to about 12 wt. %, and has a pH value in the range of about 2.0 to about 6.5, preferably about 2.5- about 5.5, particularly preferably about 2.8 to about 5.0, in each case measured at about 20° C., the wt. % data referring in each case to the weight of the hydrogen peroxide. % Data refer in each case to the weight of the oxidant preparation, and the preparation thus obtained is then applied to keratinous fibers.

Another object of the present disclosure is a cosmetic process for lightening keratinous fibers, in particular human hair, which is exemplified by contacting a portion unit as described above, wherein the oxidizing agent is selected from percarbonates, perborates or percarbamide or mixtures thereof with peroxodisulfate(s), with water or an aqueous hydrogen peroxide-free preparation, the weight ratio of the oxidizing agent to the water being preferably about 10:100 to about 28:100, more preferably about 12:100 to about 16:100, and subsequently applying the preparation thus obtained to keratinous fibers.

Preferably, the volume ratio of the oxidant to water is about 1:1 to about 1:5, more preferably about 1:2 to about 1:3. Thus, if the cosmetic product comprises only peroxodisulfate(s) as a solid oxidant, the coating is contacted with a liquid oxidant preparation as contemplated herein. If the cosmetic agent comprises percarbonates, perborates or percarbamide or mixtures of these with peroxodisulfate(s), i.e., for example, mixtures such as peroxodisulfate/percarbonate or peroxodisulfate/perborate or peroxodisulfate/percarbamide, as a solid oxidizing agent, it is brought into contact with water before use to obtain a ready-to-use cosmetic preparation. By contact with the water or the liquid oxidant preparation (M2), the coating dissolves rapidly and the ready-to-use preparation is obtained, which is not adversely affected in its effectiveness by the dissolved coating.

In a further embodiment, the problem underlying the present disclosure is solved by a packaging unit (kit-of-parts) comprising—packaged separately from each other—the following:
at least one coating comprising a cosmetic agent for lightening keratinous fibers, as previously described, and
a liquid oxidizer preparation, which is provided, for example, in a bottle.

### EXAMPLES

The present disclosure will be explained in more detail with reference to the following example, which is in no way to be construed as limiting the scope of the present disclosure.

The following bags of non-woven polyvinyl alcohol were provided:

i) cross-laid and spot-welded nonwoven fabric with a basis weight of 28 g/m²;

ii) cross-laid and spot-welded non-woven fabric with a basis weight of 38 g/m²;

(iii) cross-laid and spot-welded non-woven fabric with a basis weight of 66 g/m²;

(iv) woven non-woven fabric with a basis weight of 60 g/m²;

(v) woven non-woven fabric with a basis weight of 100 g/m²;

(vi) woven non-woven fabric with a basis weight of 140 g/m²;

and each filled with a blonding agent of the composition summarized in Table 1 (INCI names) in powder form. Suitable nonwovens are available, for example, under the trade names DO 102 and DO 105 from Freudenberg Performance Materials Holding SE & Co. KG, Germany.

TABLE 1

| Component (INCI designation, if relevant) | Quantity (wt..-%) |
|---|---|
| Ammonium persulfate + 0.5% silica | 9.6 |
| Potassium persulphate | 29.8 |
| Hydrous sodium silicate (61% SiO₂, 23% | 37.5 |
| Na₂O, 17% water) (trade name: Britesil) | |
| Thickener (acrylate copolymers, | 4.6 |
| carboxymethyl cellulose, hydroxyethyl | |
| cellulose) | |
| Magnesium carbonate | 13.2 |
| Hydrophilic fumed silica | 0.5 |
| Polyquaternium-4 | 0.3 |
| Sodium chloride | 1.0 |
| Succinic acid | 1.0 |
| Arginine | 0.5 |
| Lysine hydrochloride | 0.5 |
| Na₄EDTA powder | 1.5 |

The following oxidant preparation (M2) was provided:

TABLE 2

| Components | Quantity (wt..-%) |
|---|---|
| Water (demineralized) | 84.42 |
| EDTA-Na₂ | 0.16 |
| Disodium pyrophosphate | 0.32 |
| Na-benzoate | 0.04 |
| C16-18 fatty alcohol sulphate - Na salt | 0.18 |
| Cetearyl Alcohol | 1.73 |
| PEG-40 Castor Oil | 0.34 |
| Hydrogen peroxide (50 wt. % in water) | 12.77 |
| Phosphoric acid (85 wt. % in water) | 0.04 |

The bags comprising the preparation according to Table 1 were dissolved in the liquid oxidant preparation according to Table 2. The weight ratio was 33.3 wt. % of the composition according to Table 1 including the weight of the nonwoven fabric, and 66.7 wt. % of the oxidizer preparation. The bag as well as the powder dissolved without leaving any residue.

In another embodiment, a composition according to Table 1 was contacted with the following oxidant preparation (M2):

TABLE 3

| Components | Quantity (wt..-%) |
|---|---|
| Water (demineralized) | 62.61 |
| Sodium hydroxide (50 wt. % in water) | 0.77 |
| Dipicolinic acid | 0.10 |
| Disodium pyrophosphate | 0.03 |
| Etidronic acid (60 wt. % in water) | 1.50 |
| C12-14 fatty alcohol ether sulphate sodium | 2.00 |
| salt (2 EO, 27 wt. % in water) | |
| Hydrogen peroxide (50 wt. % in water) | 15.00 |
| Aculyn 33A* | 15.00 |

*Aculyn 33A: aqueous dispersion of Acrylates Copolymer (blend of crosslinked (meth)acrylic acid/acrylic acid C1-C6 alkyl ester copolymers); 28 wt. % polymer content (active ingredient)

The bags comprising the preparation according to Table 1 were dissolved in the liquid oxidant preparation (M2). The weight ratio was 25 wt. % of the composition according to Table 1 including the weight of the nonwoven, and 75 wt. % of the oxidizer preparation according to Table 3. The bag as well as the powder dissolved without leaving any residue.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A portion unit comprising at least one receiving chamber formed by a water-soluble envelope, wherein the water-soluble envelope comprises a water-soluble nonwoven fabric and wherein the receiving chamber comprises a cosmetic agent for lightening keratinous fibers.

2. A portion unit according to claim 1, wherein the water-soluble nonwoven is selected from the group of needle punched nonwovens.

3. A portion unit according to claim 1, wherein the water-soluble nonwoven fabric is a non-woven, cross-laid and spot-sealed nonwoven fabric.

4. A portion unit according to claim 1, wherein the water-soluble nonwoven has a basis weight of about 35 to about 160 g/m².

5. A portion unit according to claim 1, wherein the water-soluble nonwoven comprises at least about 50 wt. % of polyvinyl alcohol or polyvinyl alcohol polymers based on the total weight of the water-soluble coating.

6. A portion unit according to claim 1, wherein the cosmetic composition comprises at least one hair-lightening oxidizing agent selected from peroxodisulfates, percarbonates, perborates, percarbamides, and combinations thereof.

7. A portion unit according to claim 1, wherein the cosmetic composition comprises, based on its total weight, from about 10 to about 90 wt. % of peroxodisulfate(s).

8. A portion unit according to claim 7, wherein the peroxodisulfate(s) are selected from alkali metal and ammonium peroxodisulfates, and mixtures thereof.

9. A cosmetic process for lightening keratinous fibers, wherein a portion unit according to claim 1 is brought into contact with a liquid oxidant preparation (M2), the liquid oxidant preparation comprising about 40 to about 96 wt. % of water and comprising hydrogen peroxide in a total amount of about 0.5 to about 23 wt. %, each based on a weight of the oxidizing agent preparation (M2) and having a pH value in the range of about 2.0 to about 6.5 measured at about 20° C., and wherein the combination of the portion unit and the liquid oxidant preparation (M2) is then applied to keratinous fibers.

10. A cosmetic process for lightening keratinous fibers wherein a portion unit according to claim 1 is brought into contact with water or an aqueous hydrogen peroxide-free preparation, wherein the oxidizing agent is selected from percarbonates, perborates, percarbamides, or mixtures thereof with peroxodisulfate(s), and wherein the weight ratio of the oxidizing agent to the water is about 10:100 to about 28:100, and the combination of the portion unit and the water or the aqueous hydrogen peroxide-free preparation is then applied to keratinous fibers.

11. A portion unit according to claim 2, wherein the water-soluble nonwoven fabric is a non-woven, cross-laid and spot-sealed nonwoven fabric.

12. A portion unit according to claim 2, wherein the water-soluble nonwoven has a basis weight of about 35 to about 160 g/m².

13. A portion unit according to claim 1, wherein the water-soluble nonwoven comprises at least about 70 wt. % of polyvinyl alcohol or polyvinyl alcohol polymers based on the total weight of the water-soluble coating.

14. A portion unit according to claim 1, wherein the water-soluble nonwoven comprises at least about 90 wt. % of polyvinyl alcohol or polyvinyl alcohol polymers based on the total weight of the water-soluble coating.

15. A portion unit according to claim 1, wherein the water-soluble nonwoven comprises at least about 98 wt. % of polyvinyl alcohol or polyvinyl alcohol polymers based on the total weight of the water-soluble coating.

16. A portion unit according to claim 2, wherein the water-soluble nonwoven comprises at least about 70 wt. % of polyvinyl alcohol or polyvinyl alcohol polymers based on the total weight of the water-soluble coating.

17. A portion unit according to claim 2, wherein the water-soluble nonwoven comprises at least about 90 wt. % of polyvinyl alcohol or polyvinyl alcohol polymers based on the total weight of the water-soluble coating.

18. A portion unit according to claim 2, wherein the water-soluble nonwoven comprises at least about 98 wt. % of polyvinyl alcohol or polyvinyl alcohol polymers based on the total weight of the water-soluble coating.

19. A portion unit comprising according to claim 1 wherein
the water-soluble envelope consists of the water-soluble nonwoven fabric;
the water-soluble nonwoven is selected from the group of needle punched nonwovens;
the water-soluble nonwoven fabric is a single layered non-woven, cross-laid and spot-sealed nonwoven fabric;
the water-soluble nonwoven has a basis weight of about 35 to about 160 g/m²;
the water-soluble nonwoven comprises at least about 98 wt. % of polyvinyl alcohols based on the total weight of the water-soluble coating;
the cosmetic composition comprises at least one hair-lightening oxidizing agent selected from peroxodisulfates, percarbonates, perborates, percarbamides, and combinations thereof;
the cosmetic composition comprises, based on its total weight, from about 30 to about 60 wt. % of peroxodisulfate(s) selected from sodium peroxodisulfate, potassium peroxodisulfate, ammonium peroxodisulfate, and combinations thereof.
